(19) United States
(12) Patent Application Publication
(10) Pub. No.: US 2025/0022538 A1
(43) Pub. Date: Jan. 16, 2025

(54) Machine Learning Techniques to Identify a Long-Read Sequence

(71) Applicant: REGENTS OF THE UNIVERSITY OF MICHIGAN, Ann Arbor, MI (US)

(72) Inventors: Brian D. Athey, Ann Arbor, MI (US); Alan Boyle, Ann Arbor, MI (US); Monica Holmes, Ann Arbor, MI (US); Gregory Farnum, Ann Arbor, MI (US)

(21) Appl. No.: 18/766,151

(22) Filed: Jul. 8, 2024

Related U.S. Application Data
(60) Provisional application No. 63/526,365, filed on Jul. 12, 2023.

(51) Int. Cl. G16B 40/10; C12Q 1/6869; G16B 30/00

(52) U.S. Cl. CPC G16B 40/10; C12Q 1/6869; G16B 30/00

(57) ABSTRACT

To train a machine learning algorithm to identify DNA sequences, an electrochemical sensor detects a set of electrical signals from each of a plurality of standards each representing a known sequence. A computing device obtains the sets of electrical signals and trains a machine learning model to identify a sequence using, for each of the standards, (i) characteristics of the set of electrical signals, and (ii) the known sequence of the standard. The electrochemical sensor detects a set of electrical signals from a DNA sequence from a patient having an unknown sequence. The computing device obtains the set of electrical signals from the patient DNA sequence and applies characteristics of the set of electrical signals from the patient DNA sequence to the trained machine learning model to identify a sequence for the patient DNA sequence.

### CROSS-REFERENCE TO RELATED APPLICATIONS

This application claims priority to and the benefit of the filing date of provisional U.S. Application Ser. No. 63/526,365, filed on Jul. 12, 2023, entitled “Machine Learning Techniques to Identify a Long-Read Sequence,” the entire disclosure of which is hereby expressly incorporated by reference herein.

### FIELD OF THE INVENTION

The present application relates to identifying DNA sequences such as pharmacogene sequences and, more specifically, to a method and system for utilizing machine learning techniques to identify a long-read sequence for a patient without using polymerase chain reaction (PCR).

### BACKGROUND

A pharmacogenomic assay is a type of genetic test that analyzes a person's DNA to identify genetic variations in pharmacogenes that affects their response to medication. Accurately detecting genomic variants, including small and large structural variations, single nucleotide variants, and copy number variants, is crucial for effective pharmacogenomic testing. False positive and false negative test results can misinform physicians using pharmacogenetic (PGx) testing to guide medication-based treatment, resulting in the patient experiencing an adverse drug reaction (ADR).

Due to the complex nature of pharmacogenetics, not all genetic variation is accounted for in current pharmacogenetic tests. An example of the complexity is that pharmacogenes can have a high frequency of structural variation in the population and thus require long reads to disambiguate their structure. However, currently used assays typically target a limited subset of pre-selected single nucleotide variants (SNVs). Short-read sequencing methods cannot accurately profile complex or repetitive genetic loci, which include multiple genes of high pharmacogenomic relevance, such as cytochrome P450 family 2 subfamily B member 6 (CYP2B6), cytochrome P450 family 2 subfamily D member 6 (CYP2D6), and human leukocyte antigens (HLA).

Furthermore, current genetic testing typically uses PCR amplification to select genomic regions. However, polymerases are known to introduce substitutions, insertions, and deletions at significant rates throughout amplification leading to inaccuracies. Moreover, PCR or hybridization-based capture methods cannot unambiguously resolve any repeated region greater than 2,000 base pairs. However, many important pharmacogenes are longer than 2,000 base pairs. For example, CYP2D6, is 4,300 base pairs in length. When including the pseudo genes CYP2D7 and CYP2D8, the entire locus is 35,000 base pairs in length.

### SUMMARY

To provide an accurate system for performing long-read sequencing of DNA, including, but not limited to, genes such as pharmacogenes, a basecalling system uses individual DNA standards or fragments with known sequences that each include part or all of a particular DNA sequence (e.g., CYP2D6). The basecalling system clones each fragment into a bacterial plasmid to create a plasmid standard. Then the basecalling system propagates each standard in a clonal bacterial colony and determines the sequence for each standard.

The basecalling system then uses the standards and known sequences for each standard as training data to train a machine learning algorithm to identify a sequence for a patient pharmacogene. More specifically, the basecalling system provides the standards to an electrochemical sensor, such as a nanopore sensor, that converts chemical energy from a standard into a set of electrical signals. For example, the set of electrical signals may be a series of electrical current values over time which can be mapped to the base pairs (e.g., AATGCA, etc.) in the known sequence.

The basecalling system then trains the machine learning model using the set of electrical signals and known sequence for each standard. In some implementations, the basecalling system also trains the machine learning model using other characteristics of each standard, such as a start position tag of the standard indicating an initial genetic locus where the standard begins (e.g., 11q1.4), an end position tag of the standard indicating a final genetic locus where the standard ends (e.g., 11q2.1), a length tag of the standard (e.g., 100 B), a location tag of the standard (e.g., the long arm of chromosome ten, band 1), metadata regarding the origin of the sequencing data for the standard, the purity of the known sequence for the standard, the segmentation process applied for the standard, etc.

After the training period, the basecalling system may obtain a patient's DNA sequence (e.g., a pharmacogene) having an unknown sequence for example, when the patient has a sequence that does not match the signal profile in a sequence database. For example, the basecalling system may collect a sample from the patient and cut a pharmacogene from the sample using a nuclease system such as a CRISPR-Cas9 system. Then the basecalling system may provide the pharmacogene to the electrochemical sensor to detect a set of electrical signals for the patient pharmacogene. Then the basecalling system may apply characteristics of the set of electrical signals for the patient pharmacogene to the machine learning model to identify a long-read sequence for the patient pharmacogene.

In this manner, the basecalling system can detect mutations in the patient pharmacogene that are associated with pharmacological phenotypes. The basecalling system can then determine for example, whether the patient is likely to have an adverse reaction to a drug, the efficacy of the drug on the patient, a recommended dosage of the drug for the patient, etc.

In addition to identifying long-read sequences for pharmacogenes, the basecalling system may identify sequences for other types of genes or DNA sequences, such as human cancer genes (e.g., tumor protein p53 (TP53), breast cancer type 1 susceptibility protein (BRCA1), breast cancer type 2 susceptibility protein (BRCA2), epidermal growth factor receptor (EGFR), Kirsten rat sarcoma viral oncogene homolog (KRAS)), bacterial genes (e.g., Ribosomal RNA operons, DNA gyrase subunit A (gyrA), Recombinase A (recA), RNA polymerase subunit B (rpoB), Methicillin resistance gene in Staphylococcus aureus (mecA)), regular human genes (e.g., Beta-actin (ACTB), Glyceraldehyde 3-phosphate dehydrogenase (GAPDH), Beta-2-microglobulin (B2M), Hemoglobin subunit beta (HBB), Cystic fibrosis transmembrane conductance regulator (CFTR)), plant genes (e.g., Repressor of GA signaling (RGA), Fatty acid desaturase 2 (FAD2), High-affinity potassium transporter 1 (HKT1), WRKY transcription factors (WRKY), Myeloblastosis oncogene homolog transcription factors (MYB)), viral genes (e.g., Large protein (L) (in Ebola virus), Envelope protein (E) (in Dengue virus), Polymerase basic protein 2 (PB2) (in Influenza virus), Glycoprotein B (gB) (in Herpes Simplex Virus), Nucleocapsid protein (N) (in Coronavirus)), fungal genes (e.g., Internal transcribed spacer region (ITS) (for species identification), Glucan synthase (FKS1) (associated with echinocandin resistance), Ergosterol biosynthesis gene (ERG11), Beta-tubulin (TUB2), Sterol 14α-demethylase (CYP51)), immune system genes (e.g., Human leukocyte antigen genes (HLA), T-cell receptor genes (TCR), Immunoglobulin heavy chain genes (IGH), Cluster of Differentiation 3 (CD3), NOD-like receptors (NLR)), etc.

Additionally, by training the machine learning model using known sequences for various standards, the basecalling system can more accurately identify long-read sequences for patients. This enables higher accuracy phasing calls (particularly with copy number variants (CNVs), insertions, deletions, and duplications) by unambiguously resolving compound heterozygosity.

In alternative machine learning systems, the true sequence that corresponds to each signal is typically unknown. Instead, the sequences used as labels for the machine learning model are estimated using a process where an existing basecaller is used to generate a sequence, which is then aligned to a reference genome to estimate the true sequence. This process can introduce errors into the labels, which can complicate the training and evaluation of new basecallers. If a new basecaller is evaluated using labels derived from an existing basecaller's predictions, the measured accuracy can be artificially high because any errors made by the initial basecaller that are repeated by the new basecaller will not be detected. The new basecaller may indirectly learn the errors of the previous one. Accordingly, the basecalling system described herein may be used to retrain or fine-tune existing basecallers to obtain a higher accuracy.

In an embodiment, a method for training a machine learning algorithm to identify DNA sequences is provided. The method includes for each of a plurality of standards each representing a known sequence, detecting, by an electrochemical sensor, a set of electrical signals from each standard. The method further includes training a machine learning model to identify a sequence using, for each of the plurality of standards, (i) characteristics of the set of electrical signals, and (ii) the known sequence of the standard.

In another embodiment, a computing device for training a machine learning algorithm to identify DNA sequences is provided. The computing device includes one or more processors, and a non-transitory computer-readable memory coupled to the one or more processors and storing instructions thereon. When executed by the one or more processors, the instructions cause the computing device to train a machine learning model to identify a sequence using, for each of a plurality of standards each representing a known sequence, (i) characteristics of a set of electrical signals from the standard, and (ii) the known sequence of the standard.

In yet another embodiment, a system for training a machine learning algorithm to identify DNA sequences is provided. The system includes an electrochemical sensor configured to detect electrical signals from a sample and a computing device. The computing device includes one or more processors, and a non-transitory computer-readable memory coupled to the one or more processors and storing instructions thereon. When executed by the one or more processors, the instructions cause the computing device to obtain, from the electrochemical sensor, a plurality of sets of electrical signals each corresponding to one of a plurality of standards each representing a known sequence. The instructions also cause the computing device to train a machine learning model to identify a sequence using, for each of the plurality of standards, (i) characteristics of the set of electrical signals, and (ii) the known sequence of the standard.

### BRIEF DESCRIPTION OF THE DRAWINGS

FIG. 1 illustrates a block diagram of a computer network and system on which an exemplary basecalling system may operate in accordance with the presently described embodiments;

FIG. 2 depicts example characteristics of plasmid standards and their known sequences that may be provided as training data to a machine learning model;

FIG. 3 depicts a combined block and logic diagram that depicts the generation of a long-read sequence for a patient pharmacogene using a machine learning model;

FIG. 4 depicts an example sequence diagram that depicts each of the processes performed to identify a long-read sequence for a pharmacogene from a patient's biological sample; and

FIG. 5 depicts a flow diagram representing an exemplary method for identifying a long-read sequence for a patient pharmacogene using machine learning techniques in accordance with the presently described embodiments.

### DETAILED DESCRIPTION

Although the following text sets forth a detailed description of numerous different embodiments, it should be understood that the legal scope of the description is defined by the words of the claims set forth at the end of this disclosure. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

Accordingly, as used herein, the term “health care provider” may refer to any provider of medical or health services. For example, a health care provider may be a physician, clinician, nurse practitioner, physician assistant, an insurer, a pharmacist, a hospital, a clinical facility, a pharmacy technician, a pharmaceutical company, a research scientist, other medical organization or medical professional licensed to prescribe medical products and medicaments to patients, etc.

As used herein, the term “patient” may refer to any human or other organism, or combination thereof, whose health, longevity, or other medical outcomes is the object of clinical or research interest, investigation, or effort. The term “subject” may refer to any human or other organism, or combination thereof, whose biological characteristics are the object of research interest.

Additionally, as used herein, the term “pharmacogene” may refer to a gene that is involved in the metabolism of, or response to, drugs in the human body, such as SLCO1B1, NUDT15, DPYD, CYP4F2, CYP1A2, CYP2B6, CYP2C19, CYP2C9, CYP2D6, CYP2A6, CYP3A4, CYP3A5, CFTR, UGT2B15, UGTA4, HTR2A, MC4R, UGT2B15, ADRA2A, ABCB1, COMT, HLA-A, and HLA-B.

A “CRISPR/Cas system” is a nuclease system comprising a CRISPR-associated (Cas) endonuclease (or engineered endonuclease based on a Cas endonuclease) and a guide RNA. A “CRISPR/Cas system” includes, but is not limited to, a CRISPR/Cas9 system. Other exemplary CRISPR/Cas systems comprise different Cas proteins such as Cas12a.

As used herein the term “standard” may refer to a plasmid standard created by cloning a DNA fragment of known sequence corresponding to, for example, part or all of a DNA sequence from a subject into a bacterial plasmid. Each standard may include a unique molecular barcode, allowing tracking of the known DNA sequence of each standard.

“Golden Gate cloning” or “Golden Gate assembly” as used herein may refer to a one-pot, one-step cloning procedure [Engler et al., PLoS One, 3(11): 1-7 (2008); Kirchmaier et al., PLoS One, 8(10): e76117 (2013); Engler and Marillonnet, Methods Mol Biol, 1116: 119-131 (2014);]. The method takes advantage of Type IIS restriction enzymes (e.g., BsaI), which cleave DNA outside their recognition sequences. The result is an ordered assembly of a plasmid and as many as nine DNA fragments.

A “barcode” as used herein may refer to a known nucleotide sequence included in a plasmid (which may be a plasmid standard) that is a “signal” used to classify the plasmid. The barcode, when sequenced by the sequencing technology of interest, adds no significant error to either the training process or the sample analysis process. The barcodes have a unique signal structure compared to genomic sequences in plasmids.

As used herein, the term “pharmacological phenotype” may refer to any discernible phenotype which may have bearing on medical treatment, patient longevity and outcomes, quality of life, etc., in the context of clinical care, management and finance of clinical care, and pharmaceutical and other medical and biomedical research in humans and other organisms. Such phenotypes may include pharmacokinetic (PK) and pharmacodynamic phenotypes (PD) including all phenotypes of rates and characters of absorption, distribution, metabolism, and excretion of drugs (ADME), as well as response to drugs related to efficacy, therapeutic dosages of drugs, half-lives, plasma levels, clearance rates, etc., as well as adverse drug events, adverse drug response and corresponding severities of the adverse drug events or adverse drug response, organ injury, substance abuse and dependence and the likelihood thereof, as well as body weight and changes thereof, mood and behavioral changes and disturbances. Such phenotypes may also include reactions, beneficial and adverse, to combinations of drugs, drugs interactions with genes, sociological and environmental factors, dietary factors, etc. They may also include adherence to a pharmacological or non-pharmacological treatment regime.

As used herein, the term “long-read sequence” may refer to a fragment of a genomic sequence that is longer than a “short-read sequence” which is typically between 75 and 300 bp. A long-read sequence may be 2,000 bp, 5,000 bp, 10,000 bp, 100,000 bp, 1 MB, etc.

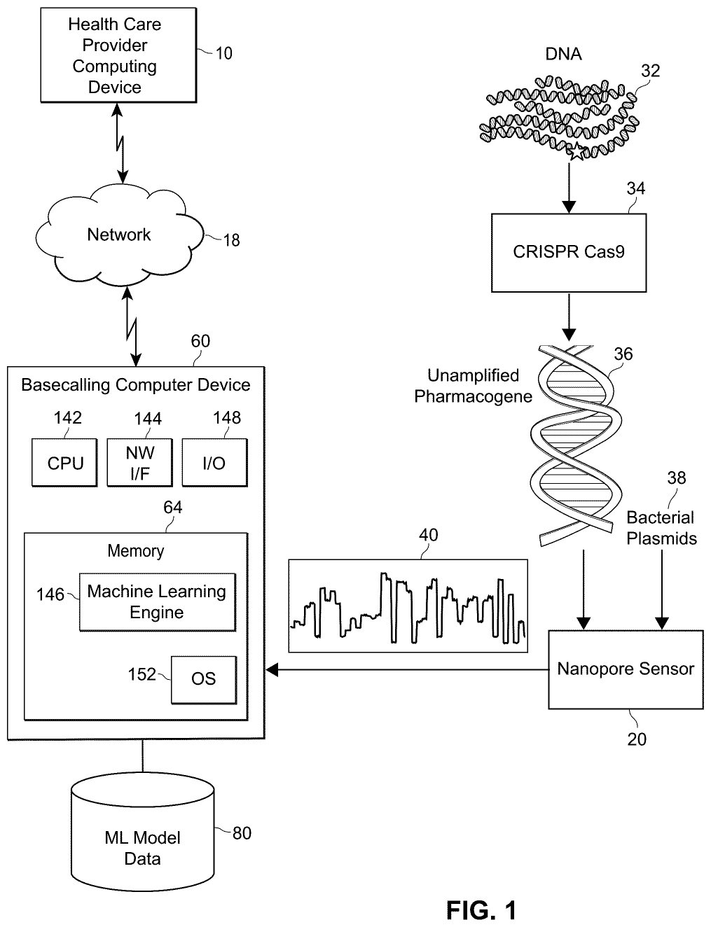

FIG. 1 illustrates an example basecalling system 100. The basecalling system 100 may include a basecalling computing device 60, a health care provider computing device 10, and an electrochemical sensor 20, such as a nanopore sensor which may be communicatively connected through a network 18, as described below. In an embodiment, the basecalling computing device 60 and the health care provider computing device 10 may communicate via wireless signals over a communication network 18, which can be any suitable local or wide area network(s) including a WiFi network, a Bluetooth network, a cellular network such as 3G, 4G, Long-Term Evolution (LTE), 5G, the Internet, etc. In some instances, the health care provider computing device 10 may communicate with the communication network 18 via an intervening wireless or wired device, which may be a wireless router, a wireless repeater, a base transceiver station of a mobile telephony provider, etc.

The health care provider computing device 10 may include, by way of example, a tablet computer, a smart watch, a network-enabled cell phone, a wearable computing device such as a smart watch, smart glasses, or a smart headset, a personal digital assistant (PDA), a mobile device smart-phone also referred to herein as a “mobile device,” a laptop computer, a desktop computer, wearable biosensors, a portable media player (not shown), a phablet, any device configured for wired or wireless RF (Radio Frequency) communication, etc. Moreover, any other suitable computing device that presents pharmacogenetic data for patients or a report based on the pharmacogenetic data may also communicate with the basecalling computing device 60.

The basecalling computing device 60 may be a cloud based server, an application server, a web server, a desktop computer, etc., and includes a memory 64, one or more processors (CPU) 142 such as a microprocessor coupled to the memory 64, a network interface unit 144, and an I/O module 148 which may be a keyboard or a touchscreen, for example.

The basecalling computing device 60 may also be communicatively connected to a machine learning model (ML) database 80. The ML database 80 may store the training data including information about the various standards, such as the start and end positions of each standard, the length of each standard, etc., characteristics of sets of electrical signals corresponding to each standard, and known sequences for each standard. The ML database 80 may also store a machine learning model generated using the training data.

The memory 64 may be tangible, non-transitory memory and may include any types of suitable memory modules, including random access memory (RAM), read only memory (ROM), flash memory, other types of persistent memory, etc. The memory 64 may store, for example instructions executable of the processors 142 for an operating system (OS) 152 which may be any type of suitable operating system such as modern smartphone operating systems, for example. The memory 64 may also store, for example instructions executable on the processors 142 for a machine learning engine 146 which may include a training module and a basecalling module. In some embodiments, the machine learning engine 146 may be a part of one or more of the health care provider computing device 10, the basecalling computing device 60, or a combination of the basecalling computing device 60 and the health care provider computing device 10.

Also in some embodiments, the training module may be executed on one computing device and the basecalling module may be executed on another computer device. For example, the training module on one computing device may train a machine learning model to identify a long-read sequence for a pharmacogene. The training module may then provide the trained machine learning model to another computing device. The basecalling module on the other computing device may then obtain a set of electrical signals from a patient's DNA sequence (e.g., a pharmacogene, a human cancer gene, a regular human gene, an immune system gene, etc.), and may apply characteristics of the set of electrical signals to the trained machine learning model to identify a long-read sequence for the patient's DNA sequence.

In any event, the machine learning engine 146 may receive a set of electrical signals 40 from the electrochemical sensor 20 when a plasmid standard passes through the electrochemical sensor 20. More specifically, the set of electrical signals 40 may include time series data indicating electrical current values (also referred to herein as “current values”) at several points in time. To detect the set of electrical signals 40, a fragment that includes all or part of a DNA sequence (e.g., CYP2D6) is cloned (e.g., using Golden Gate cloning) into a bacterial plasmid which may have an associated barcode sequence to create a plasmid standard 38. The fragment may include a wild-type allele of a pharmacogene or a mutant allele of the pharmacogene. The fragment is not amplified using PCR. The standard may include a bacterial origin of replication and a selectable marker. The standard is then propagated in a clonal bacterial colony (e.g., E. coli) and provided to the electrochemical sensor 20 to detect electrical signals from the standard. For example, the electrochemical sensor 20 may include nanopores embedded in an electro-resistant membrane. Each nanopore has its own electrode connected to a channel and sensor chip which measures the current that flows through the nanopore. The electrical signals may produce a set of “signatures” indicative of the four nucleotide bases: adenine (A), cytosine (C), guanine (G), and thymine (T).

The machine learning engine 146 also receives the known genetic sequence (e.g., the barcode) for the standard. For example, a user may enter the known genetic sequence via text input. This process may be repeated for several standards to provide the machine learning engine 146 with training data to train a machine learning model.

In some implementations, the machine learning engine 146 identifies characteristics of the sets of electrical signals and uses the characteristics as training data. For example, the characteristics may be the raw time series data of current values over time. In another example, the characteristics may include the duration of the time series data, the peak current value, the minimum current value, the average current value, the rate of change in the current values, etc.

Additionally, the characteristics may be identified over a moving time window. For example, the moving time window may be selected so that the duration of the moving time window corresponds to the time it takes for one base to pass through the electrochemical sensor 20 (e.g., 1 ns, 10 ns, 1 μs, etc.). The first moving time window may correspond to the first base in the sequence for the standard, the second moving time window may correspond to the second base in the sequence for the standard, the nth moving time window may correspond to the nth base in the sequence for the standard, etc.

The machine learning engine 146 may identify characteristics within each time window of the moving time window. The characteristics within each time window may include the set of current values within the time window, the peak current value, the minimum current value, the average current value, the change in current values over the time window, the duration within the time window where the current value is at or within a threshold range of the peak, the duration within the time window where the current value is at or within a threshold range of the minimum, or any other suitable characteristics for identifying a “signature” indicative of a nucleotide base for the standard at a location within the sequence corresponding to the time window.

In any event, the training module of the machine learning engine 146 may classify the characteristics of the sets of electrical signals into the known sequences for the standards, so that the characteristics of a set of electrical signals for a standard are “labeled” with the known sequence (e.g., the barcode) for the standard. Accordingly, the training module trains the machine learning model with a ground truth labeled training dataset that includes electrical signal characteristics for a standard labeled with the known sequence for the standard.

In some implementations, the training module may classify portions of the sets of electrical signals into portions of the known sequences. For example, the training module may classify characteristics within a time window into a corresponding nucleotide base (A, G, C or T) within the known sequence. In another example, the training module may classify characteristics of a portion of the set of electrical signals into a corresponding subset of nucleotide bases (e.g., AGTCAGTC) within the known sequence.

The training module may then analyze the labeled standards and corresponding electrical signal characteristics to generate a machine learning model for identifying DNA sequences (e.g., pharmacogene sequences). In some implementations, the training module may generate a separate machine learning model for each pharmacogene or other gene (e.g., a human cancer gene, a bacterial gene, a regular human gene, a plant gene, a viral gene, a fungal gene, an immune system gene, etc.). For example, the training module may generate a first machine learning model for identifying a long-read sequence for CYP2D6 by training the first machine learning model with standards that include part or all of CYP2D6. The start and end positions for each of the standards included in the training data may be the same for the first machine learning model. The training module may also generate a second machine learning model for identifying a long-read sequence for CYP2B6 by training the second machine learning model with standards that include part or all of CYP2B6. A third machine learning model may be generated for CYP2A6, a fourth machine learning model may be generated for CFTR, etc.

In other implementations, the training module may generate a single machine learning model using training data from standards from different DNA sequences (e.g., pharmacogenes, human cancer genes, bacterial genes, regular human genes, plant genes, viral genes, fungal genes, immune system genes, etc.) and/or include different start and/or end positions. The training module may train the machine learning model using characteristics of the standards, such as a start position tag of the standard indicating an initial genetic locus where the standard begins (e.g., 11q1.4), an end position tag of the standard indicating a final genetic locus where the standard ends (e.g., 11q2.1), a length tag of the standard (e.g., 100 B), a location tag of the standard (e.g., the long arm of chromosome ten, band 1), etc.

In any event, the set of training data may be analyzed using various machine learning techniques, such as supervised learning. These techniques may include a Hidden Markov Model (HMM) for example, where the HMM parameters are optimized using the Baum-Welch expectation maximization algorithm. The number of hidden states may be equal to the number of different sequences included in the standards. In some implementations, the hidden states of the HMM may be the different sequences and the observable states may be the electrical signal characteristics. The initial probability for a hidden state may be the likelihood that a person's pharmacogene includes a particular sequence corresponding to the hidden state. The emission probabilities for a hidden state may be likelihoods that a person's pharmacogene includes a particular sequence corresponding to the hidden state given different sets of electrical characteristics.

These techniques may also include regression algorithms (e.g., ordinary least squares regression, linear regression, logistic regression, stepwise regression, multivariate adaptive regression splines, locally estimated scatterplot smoothing, etc.), instance-based algorithms (e.g., k-nearest neighbors, learning vector quantization, self-organizing map, locally weighted learning, etc.), regularization algorithms (e.g., Ridge regression, least absolute shrinkage and selection operator, elastic net, least-angle regression, etc.), decision tree algorithms (e.g., classification and regression tree, iterative dichotomizer 3, C4.5, C5, chi-squared automatic interaction detection, decision stump, M5, conditional decision trees, etc.), clustering algorithms (e.g., k-means, k-medians, expectation maximization, hierarchical clustering, spectral clustering, mean-shift, density-based spatial clustering of applications with noise, ordering points to identify the clustering structure, etc.), association rule learning algorithms (e.g., apriori algorithm, Eclat algorithm, etc.), Bayesian algorithms (e.g., naïve Bayes, Gaussian naïve Bayes, multinomial naïve Bayes, averaged one-dependence estimators, Bayesian belief network, Bayesian network, etc.), artificial neural networks (e.g., perceptron, Hopfield network, radial basis function network, etc.), deep learning algorithms (e.g., multilayer perceptron, deep Boltzmann machine, deep belief network, convolutional neural network, stacked autoencoder, generative adversarial network, etc.), dimensionality reduction algorithms (e.g., principal component analysis, principal component regression, partial least squares regression, Sammon mapping, multidimensional scaling, projection pursuit, linear discriminant analysis, mixture discriminant analysis, quadratic discriminant analysis, flexible discriminant analysis, factor analysis, independent component analysis, non-negative matrix factorization, t-distributed stochastic neighbor embedding, etc.), ensemble algorithms (e.g., boosting, bootstrapped aggregation, AdaBoost, stacked generalization, gradient boosting machines, gradient boosted regression trees, random decision forests, etc.), reinforcement learning (e.g., temporal difference learning, Q-learning, learning automata, State-Action-Reward-State-Action, etc.), support vector machines, mixture models, evolutionary algorithms, probabilistic graphical models, etc.

In a testing phase, the training module may apply test electrical signal characteristics for test standards to the machine learning model to determine whether the sequences identified by the machine learning model match the known sequences for the test standards.

If the training module identifies the known sequences more frequently than a predetermined threshold amount or correctly identifies at least a threshold portion of each known sequence, the machine learning model may be provided to a basecalling module. On the other hand, if the training module does not identify the known sequences more frequently than the predetermined threshold amount, the training module may continue to obtain training data for further training.

The basecalling module may obtain the machine learning model and a set of electrical signals 40 for a pharmacogene 36 from a patient having an unknown sequence. To detect the set of electrical signals 40, a patient provides a biological sample 32 such as saliva, sweat, skin, blood, urine, stool, sweat, lymph fluid, bone marrow, hair, cheek cells, etc. comprising genomic DNA. The genomic DNA of the sample 32 is then cut, for example, using CRISPR-Cas9 (ref no. 34) to generate a fragment that includes all or part of a pharmacogene 36 (e.g., CYP2D6). The patient pharmacogene 36 is then provided to the electrochemical sensor 20 to detect electrical signals from the patient pharmacogene 36.

In some implementations, the machine learning engine 146 identifies characteristics of the sets of electrical signals for the patient pharmacogene 36. For example, the characteristics may be the raw time series data of current values over time. In another example, the characteristics may include the duration of the time series data, the peak current value, the minimum current value, the average current value, the rate of change in the current values, etc.

Additionally, the characteristics may be identified over a moving time window. The machine learning engine 146 may identify characteristics within each time window of the moving time window. The characteristics within each time window may include the set of current values within the time window, the peak current value, the minimum current value, the average current value, the change in current values over the time window, the duration within the time window where the current value is at or within a threshold range of the peak, the duration within the time window where the current value is at or within a threshold range of the minimum, or any other suitable characteristics for identifying a “signature” indicative of a nucleotide base for the patient pharmacogene 36 at a location within the sequence corresponding to the time window.

Then the basecalling module may apply the characteristics of the sets of electrical signals to the machine learning model to identify the long-read sequence for the patient pharmacogene 36. In some implementations, the basecalling module may also apply characteristics of the patient pharmacogene 36 to the machine learning model, such as a start position tag of the patient pharmacogene 36 indicating an initial genetic locus where the patient pharmacogene 36 begins, an end position tag of the patient pharmacogene 36 indicating a final genetic locus where the patient pharmacogene 36 ends, a length tag of the patient pharmacogene 36, a location tag of the patient pharmacogene 36, etc.

The machine learning engine 146 may then provide the identified long-read sequence for the patient pharmacogene 36 to a health care provider computing device 10 for display on a user interface of the health care provider computing device 10. The basecalling computing device 60 and/or the health care provider computing device 10 may also detect mutations in the patient pharmacogene 36 associated with pharmacological phenotypes by comparing the long-read sequence to a predetermined set of mutations within the pharmacogene associated with pharmacological phenotypes. The health care provider computing device 10 may also present the pharmacological phenotypes associated with the identified long-read sequence.

In some implementations, the machine learning engine 146 may produce the long-read sequence for the patient pharmacogene 36 in real-time as the patient pharmacogene 36 passes through the electrochemical sensor 20 or at least near real-time.

While the example illustrated in FIG. 1 refers to a patient pharmacogene 36, this is merely one example of a DNA sequence that can be analyzed by the machine learning engine 146 to identify a long-read sequence. The machine learning engine 146 may identify a long-read sequence for any suitable gene or DNA sequence, such as a pharmacogene, a human cancer gene, a bacterial gene, a regular human gene, a plant gene, a viral gene, a fungal gene, an immune system gene, etc.

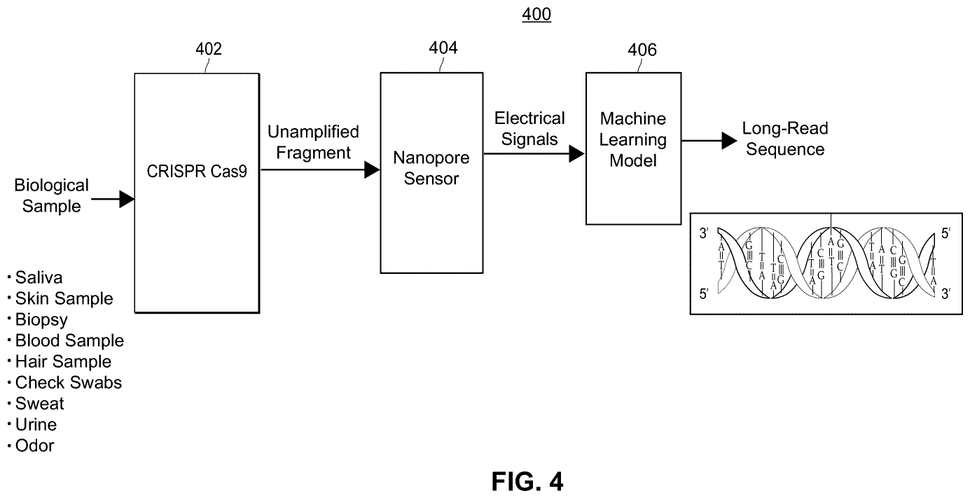

An example process 400 for identifying a long-read sequence for a pharmacogene from a patient's biological sample is illustrated in FIG. 4. As shown in FIG. 4, a patient's biological sample is collected. The patient's biological sample may include saliva, sweat, skin, blood, urine, stool, sweat, lymph fluid, bone marrow, hair, cheek cells, etc. comprising genomic DNA. The genomic DNA of the biological sample is then cut, for example, using CRISPR-Cas9 (ref no. 402) to generate an unamplified fragment that includes all or part of a pharmacogene (e.g., CYP2D6). The patient pharmacogene is then provided to a nanopore sensor 404 to detect electrical signals from the patient pharmacogene. Then the electrical characteristics and/or characteristics of the electrical signals are provided to a trained machine learning model 406 using a labeled ground truth training data set to identify a long-read sequence for the patient pharmacogene based on the electrical signals. While the process 400 illustrated in FIG. 4 includes cutting the genomic DNA of the biological sample using CRISPR-Cas9 to generate an unamplified fragment that includes a pharmacogene, the genomic DNA of the biological sample may be prepared in other ways and provided to the nanopore sensor 404. For example, a segment of the patient's genomic DNA corresponding to the pharmacogene may be cut using a restriction enzyme or different genome editing system/tool (e.g., a different CRISPR/Cas system, a transcription activator-like effector nuclease (TALEN), a zinc-finger nuclease (ZFN), or a meganuclease) and provided to the nanopore sensor 404.

While the example illustrated in FIG. 4 refers to a pharmacogene, this is merely one example of a DNA sequence that can be analyzed using the process 400 to identify a long-read sequence. The process 400 may identify a long-read sequence for any suitable gene or DNA sequence, such as a pharmacogene, a human cancer gene, a bacterial gene, a regular human gene, a plant gene, a viral gene, a fungal gene, an immune system gene, etc.

Turning back to FIG. 1, the basecalling computing device 60 may communicate with the health care provider computing device 10 via the network 18. The digital network 18 may be a proprietary network, a secure public Internet, a virtual private network and/or some other type of network, such as dedicated access lines, plain ordinary telephone lines, satellite links, combinations of these, etc. Where the digital network 18 comprises the Internet, data communication may take place over the digital network 18 via an Internet communication protocol.

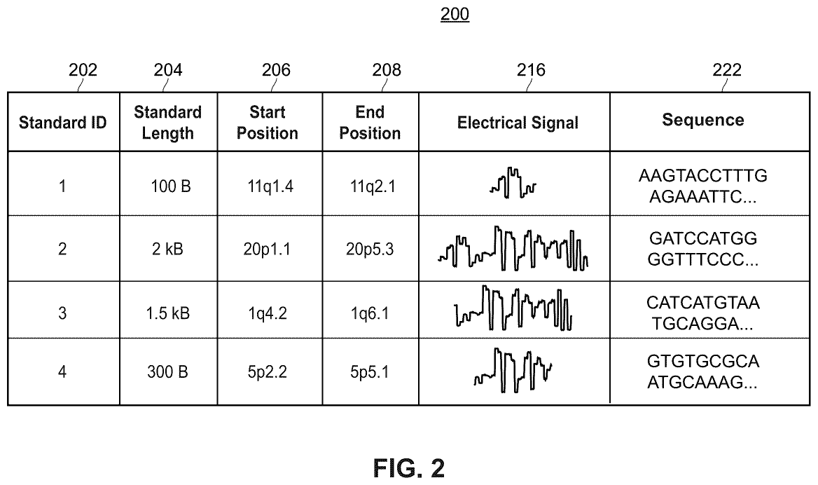

FIG. 2 illustrates example training data 200 which may be provided to the machine learning engine 146. More specifically, for each of several standards, the training data 200 includes a set of electrical signals for the standard 216, other characteristics of the standard 202-208, such as an identifier of the standard 202, a length of the standard 204, a start position for the standard 206, and an end position for the standard 208, and a known sequence 222 for the standard.

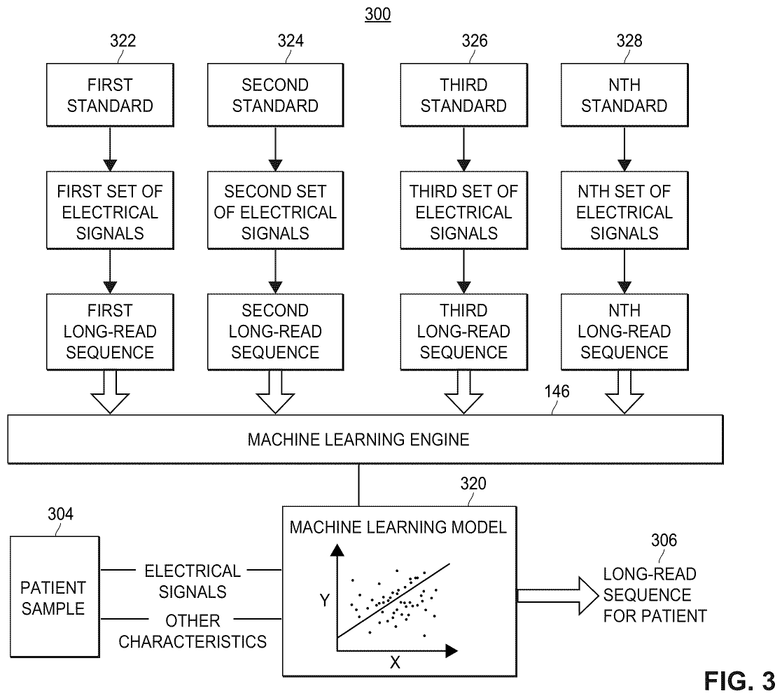

FIG. 3 schematically illustrates how the machine learning engine 146 of FIG. 1 determines the long-read sequence for a patient pharmacogene in an example scenario. Some of the blocks in FIG. 3 represent hardware and/or software components (e.g., block 146), other blocks represent data structures or memory storing these data structures, registers, or state variables (e.g., blocks 304, 312, 320), and other blocks represent output data (e.g., block 306). Input signals are represented by arrows labeled with corresponding signal names.

The machine learning engine 146 of FIG. 3 may generate the machine learning model 320. To generate the machine learning model 320, the machine learning engine 146 receives training data including an indication of a first standard 322 that has a first set of electrical signals and a known first sequence. The indication may also include metadata regarding the origin of the sequencing data, the purity of the known sequence, the segmentation process applied, etc. The training data also includes an indication of a second standard 324 that has a second set of electrical signals and a known second sequence. Furthermore, the training data includes an indication of a third standard 326 that has a third set of electrical signals and a known third sequence. Still further, the training data includes an indication of an nth standard 328 that has an nth set of electrical signals and a known nth sequence.

While the example training data includes indications of four standards 322-328, this is merely an example for ease of illustration only. The training data may include any number of standards.

The machine learning engine 146 then analyzes the training data to generate a machine learning model 320 for identifying a long-read sequence for a patient pharmacogene. While the machine learning model 320 is illustrated as a linear regression model, the machine learning model may be another type of regression model such as a logistic regression model, a decision tree, several decision trees, a neural network, a hyperplane, a Hidden Markov Model, or any other suitable machine learning model.

In any event, the system of FIG. 3 obtains a set of electrical signals for a patient pharmacogene 304 having an unknown sequence. The system may obtain characteristics of the electrical signals and/or characteristics of the patient pharmacogene 304, such as a start position tag of the patient pharmacogene 304 indicating an initial genetic locus where the patient pharmacogene 304 begins, an end position tag of the patient pharmacogene 046 indicating a final genetic locus where the patient pharmacogene 304 ends, a length tag of the patient pharmacogene 304, a location tag of the patient pharmacogene 304, etc.

The machine learning engine 146 may then apply the characteristics of the electrical signals and/or the patient pharmacogene 304 to the machine learning model 320 to identify a long-read sequence 306 for the patient pharmacogene 304. The machine learning engine 146 may then store an indication of the patient pharmacogene 304 (e.g., the electrical signal characteristics and/or other characteristics) and the identified long-read sequence 306 in the database 80.

While the example illustrated in FIG. 3 refers to a pharmacogene 304, this is merely one example of a DNA sequence that can be analyzed by the machine learning engine 146 to identify a long-read sequence. The machine leaning engine 146 may identify a long-read sequence for any suitable gene or DNA sequence, such as a pharmacogene, a human cancer gene, a bacterial gene, a regular human gene, a plant gene, a viral gene, a fungal gene, an immune system gene, etc.

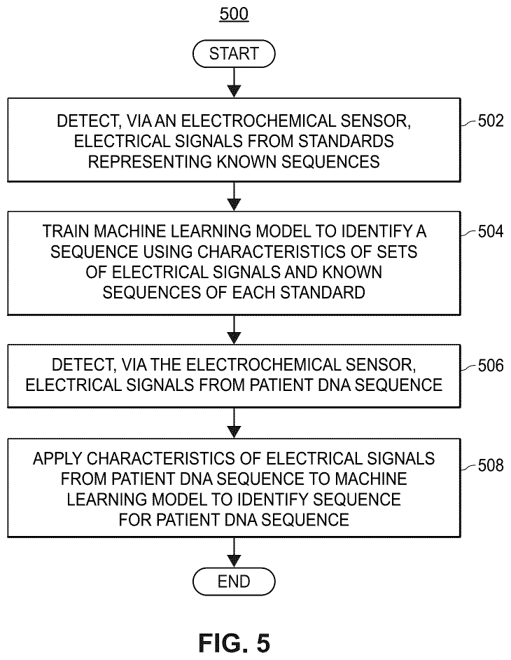

FIG. 5 illustrates a flow diagram representing an exemplary method 500 for identifying a long-read sequence for a patient DNA sequence (e.g., a pharmacogene, a human cancer gene, a regular human gene, an immune system gene, etc.) using machine learning techniques. The method 500 may be executed on the basecalling computing device 60. In some embodiments, the method 500 may be implemented in a set of instructions stored on a non-transitory computer-readable memory and executable on one or more processors on the basecalling computing device 60. For example, the method 500 may be performed by the machine learning engine 146 of FIG. 1.

At block 502, the basecalling computing device 60 detects, via an electrochemical sensor 20, electrical signals from standards having known sequences. For example, the basecalling computing device 60 may receive the electrical signals from the electrochemical sensor 20 when a plasmid standard passes through the electrochemical sensor 20. More specifically, the set of electrical signals 40 may include time series data indicating electrical current values at several points in time.

To detect the set of electrical signals, a fragment that includes all or part of a DNA sequence, such as a pharmacogene (e.g., CYP2D6) is cloned into a bacterial plasmid having an associated barcode sequence to create a plasmid standard. The fragment may include a wild-type allele of the pharmacogene or a mutant allele of the pharmacogene. The standard may include a bacterial origin of replication and a selectable marker. The standard is then propagated in a clonal bacterial colony and provided to the electrochemical sensor 20 to detect electrical signals from the standard. This process may be repeated for tens, hundreds, thousands, or any suitable number of standards.

The basecalling computing device 60 also receives a known genetic sequence for each standard. For example, a user may enter the known genetic sequence via text input. In some implementations, the basecalling computing device 60 identifies characteristics of the sets of electrical signals and uses the characteristics as training data. For example, the characteristics may be the raw time series data of current values over time. In another example, the characteristics may include the duration of the time series data, the peak current value, the minimum current value, the average current value, the rate of change in the current values, etc.

Additionally, the characteristics may be identified over a moving time window. For example, the moving time window may be selected so that the duration of the moving time window corresponds to the time it takes for one base to pass through the electrochemical sensor 20 (e.g., 1 ns, 10 ns, 1 μs, etc.).

Then at block 504, the basecalling computing device 60 trains a machine learning model using training data to identify a sequence (e.g., a long-read sequence) for a DNA sequence (e.g., a pharmacogene, a human cancer gene, a bacterial gene, a regular human gene, a plant gene, a viral gene, a fungal gene, an immune system gene, etc.). The training data may include electrical signal characteristics for each standard and the known sequence for each standard as a ground truth labeled training dataset. The training data may also include characteristics of the standards, such as a start position tag of the standard indicating an initial genetic locus where the standard begins (e.g., 11q1.4), an end position tag of the standard indicating a final genetic locus where the standard ends (e.g., 11q2.1), a length tag of the standard (e.g., 100 B), a location tag of the standard (e.g., the long arm of chromosome ten, band 1), etc.

The basecalling computing device 60 may generate a separate machine learning model for each pharmacogene, gene (e.g. a human cancer gene, a bacterial gene, a regular human gene, a plant gene, a viral gene, a fungal gene, an immune system gene, etc.), and/or genetic location. For example, the basecalling computing device 60 may generate a first machine learning model for identifying a long-read sequence for CYP2D6 by training the first machine learning model with standards comprising part or all of CYP2D6. The basecalling computing device 60 may also generate a second machine learning model for identifying a long-read sequence for CYP2B6 by training the second machine learning model with standards that comprise part or all of CYP2B6. In other implementations, the basecalling computing device 60 may generate a single machine learning model using training data from standards that include different pharmacogenes or genes and/or include different start and/or end positions.

The basecalling computing device 60 may train the machine learning model(s) using any suitable machine learning techniques, such as HMMs, regression algorithms, decision tree algorithms, Bayesian algorithms, deep learning algorithms, etc.

At block 506, the basecalling computing device 60 detects, via an electrochemical sensor 20, electrical signals from a DNA sequence of a patient (e.g., a pharmacogene, a human cancer gene, a regular human gene, an immune system gene, etc.). For example, the basecalling computing device 60 may receive the electrical signals from the electrochemical sensor 20 when the patient DNA sequence passes through the electrochemical sensor 20.

To detect the set of electrical signals, a patient provides a biological sample such as saliva, sweat, skin, blood, urine, stool, sweat, lymph fluid, bone marrow, hair, cheek cells, etc. comprising genomic DNA. In some implementations, the genomic DNA of the patient sample is cut using CRISPR-Cas9 to generate a fragment that includes all or part of the patient DNA sequence (e.g., a patient pharmacogene). The patient pharmcogene is then provided to the electrochemical sensor 20 to detect electrical signals from the standard. In other implementations, a segment of the patient's genomic DNA corresponding to the pharmacogene may be cut using a restriction enzyme or different genome editing system/tool (e.g., a different CRISPR/Cas system, a transcription activator-like effector nuclease (TALEN), a zinc-finger nuclease (ZFN), or a meganuclease) and provided to the nanopore sensor 404.

In some implementations, the basecalling computing device 60 identifies characteristics of the sets of electrical signals. For example, the characteristics may be the raw time series data of current values over time. In another example, the characteristics may include the duration of the time series data, the peak current value, the minimum current value, the average current value, the rate of change in the current values, etc.

Additionally, the characteristics may be identified over a moving time window. For example, the moving time window may be selected so that the duration of the moving time window corresponds to the time it takes for one base to pass through the electrochemical sensor 20 (e.g., 1 ns, 10 ns, 1 μs, etc.).

Then at block 508, the basecalling computing device 60 applies characteristics of the sets of electrical signals to the machine learning model to identify the long-read sequence for the patient DNA sequence. In some implementations, the basecalling module may also apply characteristics of the patient DNA sequence to the machine learning model, such as a start position tag of the patient DNA sequence indicating an initial genetic locus where the patient DNA sequence begins, an end position tag of the patient DNA sequence indicating a final genetic locus where the patient DNA sequence ends, a length tag of the patient DNA sequence 36, a location tag of the patient DNA sequence, etc.

The basecalling computing device 60 may then provide the identified long-read sequence for the patient DNA sequence to a health care provider computing device 10 for display on a user interface of the health care provider computing device 10.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as providing examples only and does not describe every possible embodiment, as describing every possible embodiment would be impractical, if not impossible. One could implement numerous alternate embodiments, using either current technology or technology developed after the filing date of this application.

We claim:

1. A computer-implemented method for training a machine learning algorithm to identify deoxyribonucleic acid (DNA) sequences, the method comprising:
for each of a plurality of standards each representing a known sequence:
detecting, by an electrochemical sensor, a set of electrical signals from each standard; and
training, by one or more processors, a machine learning model to identify a sequence using, for each of the plurality of standards, (i) characteristics of the set of electrical signals, and (ii) the known sequence of the standard.

2. The computer-implemented method of claim 1, further comprising for a DNA sequence from a patient having an unknown sequence:
detecting, by the electrochemical sensor, a set of electrical signals from the patient DNA sequence; and
applying, by the one or more processors, characteristics of the set of electrical signals from the patient DNA sequence to the machine learning model to identify a sequence for the patient DNA sequence.

3. The computer-implemented method of claim 1, further comprising:
preparing each of the plurality of standards having known sequences by:
cloning a fragment of known sequence comprising part or all of a DNA sequence into a bacterial plasmid having an associated barcode sequence to create a plasmid standard, and
propagating the standard in a clonal bacterial colony.

4. The computer-implemented method of claim 3, wherein the fragment is not amplified using polymerase chain reaction (PCR).

5. The computer-implemented method of claim 3, wherein the DNA sequence is a pharmacogene, and the fragment is a fragment of a wild-type allele of the pharmacogene or a mutant allele of the pharmacogene.

6. The computer-implemented method of claim 2, wherein the sequence for the patient DNA sequence is a long-read sequence.

7. The computer-implemented method of claim 6, wherein the patient DNA sequence is a pharmacogene of the patient, and further comprising:
detecting one or more mutations in the patient pharmacogene that are associated with one or more pharmacological phenotypes using the identified long-read sequence for the patient.

8. The computer-implemented method of claim 1, wherein for each of the standards, the machine learning model is further trained using a start position tag of the standard, an end position tag of the standard, a location tag of the standard, or a length tag of the standard.

9. The computer-implemented method of claim 1, wherein the electrochemical sensor is a nanopore sensor.

10. A computing device for training a machine learning algorithm to identify deoxyribonucleic acid (DNA) sequences, the computing device comprising:
one or more processors; and
a non-transitory computer-readable memory coupled to the one or more processors and storing thereon instructions that, when executed by the one or more processors, cause the computing device to:
train a machine learning model to identify a sequence using, for each of a plurality of standards each representing a known sequence, (i) characteristics of a set of electrical signals from the standard, and (ii) the known sequence of the standard.

11. The computing device of claim 10, further comprising:
for a DNA sequence from a patient having an unknown sequence:
apply characteristics of a set of electrical signals from the patient DNA sequence to the machine learning model to identify a sequence for the patient DNA sequence.

12. The computing device of claim 11, wherein the patient DNA sequence is not amplified using polymerase chain reaction (PCR).

13. The computing device of claim 11, wherein the patient DNA sequence is a pharmacogene of the patient, and the instructions further cause the computing device to:
detect one or more mutations in the patient pharmacogene that are associated with one or more pharmacological phenotypes using the identified sequence for the patient.

14. The computing device of claim 11, wherein the sequence of the patient DNA sequence is a long-read sequence.

15. The computing device of claim 10, wherein for each of the standards, the machine learning model is further trained using a start position tag of the standard, an end position tag of the standard, a location tag of the standard, or a length tag of the standard.

16. A system for training a machine learning algorithm to identify deoxyribonucleic acid (DNA) sequences, the system comprising:
an electrochemical sensor configured to detect electrical signals from a biological sample; and
a computing device including:
one or more processors; and
a non-transitory computer-readable memory coupled to the one or more processors and storing thereon instructions that, when executed by the one or more processors, cause the computing device to:
obtain, from the electrochemical sensor, a plurality of sets of electrical signals each corresponding to one of a plurality of standards each representing a known sequence; and
train a machine learning model to identify a sequence using, for each of the plurality of standards, (i) characteristics of the set of electrical signals, and (ii) the known sequence of the standard.

17. The system of claim 16, wherein the instructions further cause the computing device to:
obtain, from the electrochemical sensor, a set of electrical signals from a DNA sequence for a patient having an unknown sequence; and
apply characteristics of the set of electrical signals from the patient DNA sequence to the machine learning model to identify a sequence for the patient DNA sequence.

18. The system of claim 17, wherein the patient DNA sequence is not amplified using polymerase chain reaction (PCR).

19. The system of claim 17, wherein the sequence for the patient DNA sequence is a long-read sequence.

20. The system of claim 16, wherein for each of the standards, the machine learning model is further trained using a start position tag of the standard, an end position tag of the standard, a location tag of the standard, or a length tag of the standard.
